(19) United States
(12) Patent Application Publication
(10) Pub. No.: US 2009/0099633 A1
(43) Pub. Date: Apr. 16, 2009

(54) SINGLE USE ICE PACK

(75) Inventors: Michael L. Bowen, Mansfield, TX (US); William G. Mortensen, Dallas, TX (US)

Correspondence Address: Glast, Phillips & Murray, P.C, 13355 Noel Road, Suite 2200, Dallas, TX, 75240, US

(73) Assignee: ADVANCED MATERIALS GROUP, INC., Dallas, TX (US)

(21) Appl. No.: 12/337,117

(22) Filed: Dec. 17, 2008

Related U.S. Application Data
(63) Continuation-in-part of application No. 11/196,216, filed on Aug. 3, 2005, now abandoned.
(60) Provisional application No. 60/602,591, filed on Aug. 18, 2004.

(51) Int. Cl. A61F 7/00

(52) U.S. Cl. 607/114

(57) ABSTRACT

An ice pack including a bag having a containment section with an opening. Two layers of adhesive material are be disposed on adjacent portions of the opening. One layer has a lesser width than the other layer, so that when the layers are engaged, the layer with the greater width engages both the other layer and a portion of the interior surface of the bag. At least one removable liner and preferably two removable liners may be disposed on respective layers of the adhesive material. The ice pack may be satisfactory for a single use.

### RELATED APPLICATION

This application is a continuation-in-part of co-pending patent application Ser. No. 11/196,216, titled “Single use ice pack and method,” filed Aug. 3, 2005, which copending application claims the benefit of U.S. provisional application No. 60/602,591, filed Aug. 18, 2004. Both of the foregoing applications are incorporated by reference in their entirety.

### TECHNICAL FIELD

The present disclosure is related to ice packs, and more particularly, to ice packs having a bag and closure mechanism satisfactory for a single use.

### BACKGROUND

Ice packs are often used for cooling various objects, such as portions of a patient's body for medical reasons. Ice packs generally include a bag having a containment section with an associated opening or mouth. Ice may be inserted into the containment section of the bag through the opening. Various types of closure mechanisms and devices have previously been used to seal the opening of the bag after ice has been placed in the containment section. After sealing the opening, the associated ice pack may be applied to a desired location on a patient.

Ice packs are preferably sealed so that ice and any liquid in the containment section will not run out of the ice pack. Some closure devices associated with prior ice packs close or seal the associated containment section using components such as stoppers and clips. Such components often add additional cost to manufacture an ice pack and may limit the quantity of ice that can be placed within an associated containment section. Various types of ties have also been used to seal or close ice packs. Ties may also be used to attach an ice pack to a patient's limb or other parts of a patient. Such ties are frequently not used due to time and effort required to effectively seal an ice pack having such ties. Many ice packs that are designed for multiple uses are often discarded after the first use, particularly when the first use is in a hospital or other medical facility.

### SUMMARY

An ice pack may be formed with a bag having a first sidewall and a second sidewall sealed with each other to form a containment section and a mouth or opening communicating with the containment section. The opening preferably includes a closure mechanism compatible with a single use of the ice pack. Each sidewall may be formed with one or more layers of material.

For some applications, the sidewalls may include a first waterproof layer with a second layer, or exterior layer formed from relatively soft, water absorbent material. At least one layer of adhesive material may be disposed on a portion of the opening. For some applications, respective layers of adhesive material are preferably disposed on an interior portion of each sidewall adjacent to the opening. At least one release liner may be disposed on adhesive material proximate the opening. For some applications, a first release liner and a second release liner may be respectively disposed on a first layer and a second layer of adhesive material proximate the opening. The first and second release liners may cooperate with each other to form a funnel type device to assist with placing ice within the containment section of an associated ice pack.

Technical benefits of the ice pack disclosed here include providing a relatively low cost ice pack with a reliable, easy to use, closure mechanism. For some applications, a low cost disposable funnel-type device may be provided to assist with placing ice in the ice pack. Another benefit is that a fluid barrier may be formed that substantially reduces or eliminates problems associated with leakage of water after an ice pack has been filled with ice. Such a water leak is often a problem with conventional ice packs.

The ice pack disclosed here eliminates the need for relatively expensive clips or stoppers to close an ice pack. Eliminating hard plastic clips improves patient comfort during use of an ice pack having a soft, adhesive closure. The present ice pack eliminates ties and other types of closure mechanisms, which are difficult and time-consuming to use, particularly in a hospital emergency room. A single use ice pack may be particularly beneficial for use with patients in an isolation ward or similar facility. An associated single use or one time closure mechanism helps to prevent spread of contamination by eliminating refills at an ice machine.

Ice packs claimed in this application will improve compliance with existing hospital rules and regulations concerning infection control and disposal of potentially contaminated materials while at the same time reducing costs. Ice packs may be formed in accordance with teachings of the present disclosure at one-half to one-third of the cost of conventional ice packs having the same general size and configuration.

I disclose forming an ice pack by bonding or sealing portions of a first sidewall with a second sidewall to form a containment section having a mouth or opening. Ice may be placed in the containment section through the associated opening. At least one adhesive layer may be disposed on one of the sidewalls proximate the associated opening. The adhesive layer may contact adjacent portions of the other sidewall to form a fluid seal at the opening after ice has been added to the containment section. Or, respective adhesive layers may be disposed on each sidewall next to the associated opening after ice has been added to the containment section. The adhesive layers may contact each other, preferably also contacting a portion of the inner sidewall to assure two different sealing surfaces.

### BRIEF DESCRIPTION OF THE DRAWINGS

FIG. 1 is a schematic drawing showing an isometric view of one embodiment of the ice pack.

FIG. 2 is a schematic drawing showing an exploded isometric view with portions broken away of various layers of material associated with the ice pack of FIG. 1.

FIG. 3 is a schematic drawing in section taken along lines 3-3 of FIG. 1.

FIG. 4 is a schematic drawing showing an isometric view with portions broken away of the ice pack of FIG. 1 being filled with ice.

FIG. 5 is a schematic drawing showing an isometric view with portions broken away of the ice pack of FIG. 1 during closure after being filled with ice.

### DETAILED DESCRIPTION

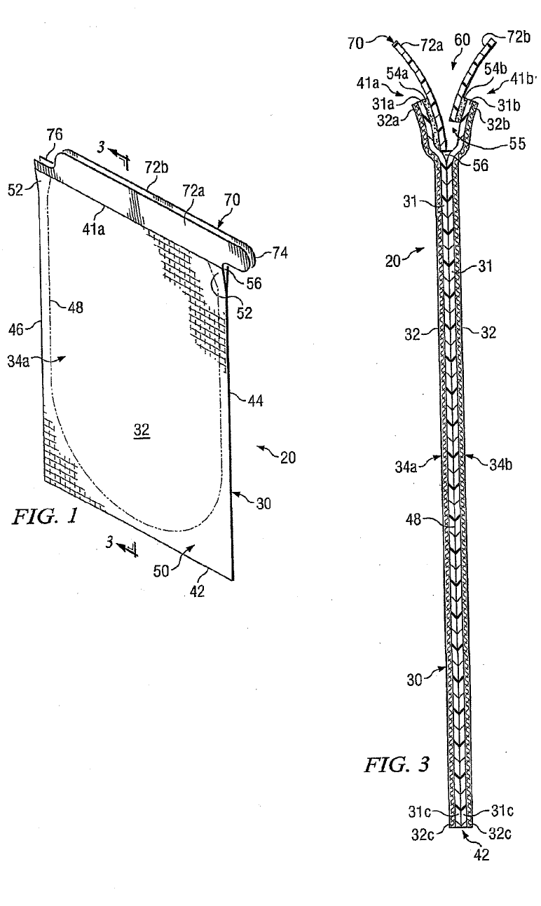

Various embodiments and their advantages are best understood by reference to FIGS. 1-5 wherein like numbers refer to same and like parts.

FIGS. 1-5 show an embodiment of the ice pack. Ice pack 20 may be satisfactory for a single or one term use to apply cold therapy to a portion of a patient's body.

For embodiments such as shown in FIGS. 1-5, the ice pack 20 may be described as having a generally rectangular configuration. However, other embodiments of the ice pack 20 may have a wide variety of other configurations (not expressly shown) such as circular, oval, square or any other appropriate configuration for applying to a patient.

The ice pack 20 may include a bag 30 defined in part by a containment section 40 and opening 60 communicating therewith. For some applications a disposable funnel type device 70 may be included as part of the ice pack 20. The ice pack 20 may include a one-time closure mechanism. A layer of adhesive material and an attached release liner, as described below, may be one example of such one time closure mechanism.

A bag 30 may be formed in part by a pair of sidewalls 34. For purposes of description, the pair of sidewalls 34 is designated in the figures as 34a and 34b. For some applications, the sidewalls 34a and 34b may be formed from the same materials with the same overall configuration and dimensions. For other applications, the sidewalls 34a and 34b may be formed with either different types of materials or with different configurations and dimensions. Other embodiments of the ice pack may be formed with each sidewall 34 having only one layer of material (not expressly shown) or with each sidewall 34 having three or more layers of material (not expressly shown)

For some applications, each sidewall 34 may include first layer or inner layer 31 and second layer or outer layer 32. For some applications, first layer 31 may be formed from a wide variety of vinyl polymers such as relatively thin, low cost polyethylene film or any other waterproof material. Metallocene based polymers may also be used. Layer 31 may also be formed from various types of generally smooth, fluid impervious plastic films.

The second layer 32 may be formed from any soft, flexible, absorbent material satisfactory for contact with a patient's skin. For example, the second layer 32 may be formed from various materials such as hydroentangled fibers and wood pulp. Polyester based fibers may also be used. Various types of commercially available woven and non-woven materials may be satisfactorily used to form the second layer 32. Relatively low cost, inexpensive materials satisfactory for forming such layers 31 and 32 are commercially available from a wide variety of manufacturers, such as Polymer Group, Inc.

For some applications, the layer 31 and the associated layer 32 may be laminated with each other prior to forming the bag 30. For example, a heat melt coating or film may be applied to one side of a layer of soft, absorbent woven or nonwoven material to produce a laminated sheet satisfactory for forming the sidewalls 34. One side of the laminated sheet may be fluid impervious. The other side of the laminated sheet may be soft and absorbent. Sheets of laminated material are commercially available from several sources.

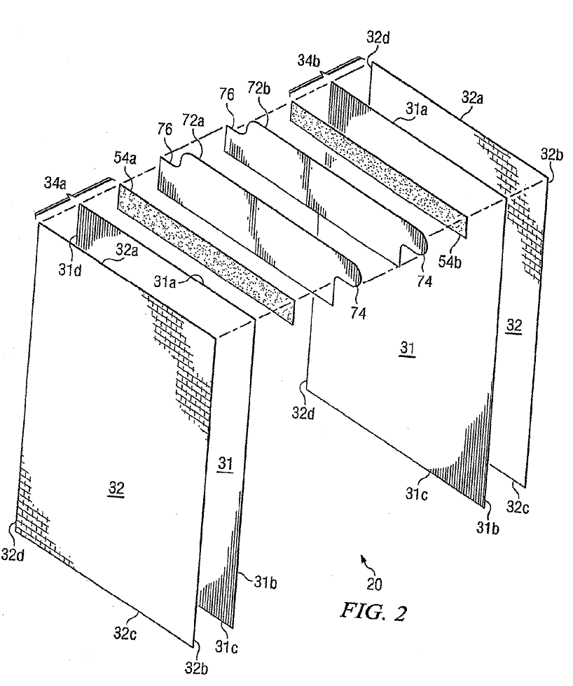

As shown in FIGS. 2 and 3, the layers 31 and 32 may have generally rectangular configurations defined in part by respective the edges 31a-31d and 32a-32d. See FIG. 2. The edges 32a and 32c for each layer 32 may extend generally parallel with each other between the edges 32b and 32d. In a similar manner, the edges 31a and 31c for each layer 31 may extend generally parallel with each other between the associated edges 31b and 31d. As previously noted for some applications, layers 31 and 32 may be laminated with each other prior to forming respective sidewalls 32a and 32b.

The bag 30 may be defined in part by a first end 41 and a second end 42 with the lateral edges 44 and 46 extending there between. See FIGS. 1 and 3. The bag 30 includes an opening 60 located at the first end 41 for use in adding ice to a containment section 40. The containment section 40 may be generally defined as a void, space or cavity formed between the sidewalls 34a, 34b, the opening 60, the respective sealed lateral edges 44, 46, and the closed end 42.

The first end 41 of bag 30 may include two portions designated 41a and 41b which cooperate with each other to provide an opening 60. The lateral edge 44 of bag 30 may be formed by bonding or sealing the edges 32b and 31b of the sidewalls 34a and 34b with each other. In a similar manner, the second end 42 or the closed end 42 of the bag 30 may be formed by bonding or sealing the edges 32c and 31c of the sidewalls 34a and 34b with each other. The lateral edge 46 may be formed by bonding or sealing the edges 32d and 31d of the sidewalls 34a and 34b with each other.

Various types of bonding and sealing techniques may be satisfactorily used to form a fluid barrier 50 along the periphery of the bag 30 adjacent to the lateral edges 44 and 46 and the end 42. For example, the lateral edges 44, 46 and the end 42 may be sealed or bonded using a heat sealing die (not expressly shown) to form the fluid barrier 50 that extends along three sides of the periphery of the bag 30. The dimensions and configuration of the fluid barrier 50 may also be as indicated in part by the dotted line 48 and the adjacent lateral edges 44, 46 and the closed end 42. The dotted line 48 also defines portions of the containment section 40. Satisfactory fluid barriers may also be formed along the periphery of the bag 30 using sonic welding techniques or RF energy bonding or both.

Respective adhesive layers 54a and 54b may be disposed on interior portions of each first layer 31 adjacent to the respective first ends 41a and 41b proximate opening 60. The adhesive layers 54 may be formed from viscous, waterproof adhesive materials capable of filling any gaps associated with sealing the edges 41a and 41b with each other and sealing with adjacent portions of the lateral edges 44 and 46. The adhesive layers 54a and 54b may be used to permanently close or seal opening 60 after filling containment section 40 with ice. Various types of acrylic based adhesive materials and rubber based adhesive materials may be satisfactorily used to form the adhesive layers 54a and 54b.

As shown in FIG. 3, the width of the second adhesive layer 54b is less than that of the first adhesive layer, thus creating an exposed region 55 where the inner surface of the corresponding first layer 31b is opposed by a portion of the first adhesive layer 54a on the opposite side of the opening 60. This allows the first adhesive layer 54a to seal to both the second adhesive layer 54b and to the inner surface of the first layer 31b in the exposed region 55. The resulting two sealing surfaces further enhance the fluid tightness of the closure.

The release liners 72a and 72b are preferably attached with respective the adhesive layers 54a and 54b. See FIGS. 2 and 3. For some applications, a single release liner may be satisfactorily used. FIG. 3 shows the second release liner 72b having a width only sufficient to cover the second adhesive layer 54b, although the second release liner 72b may have the same width as that of the first release liner 72a.

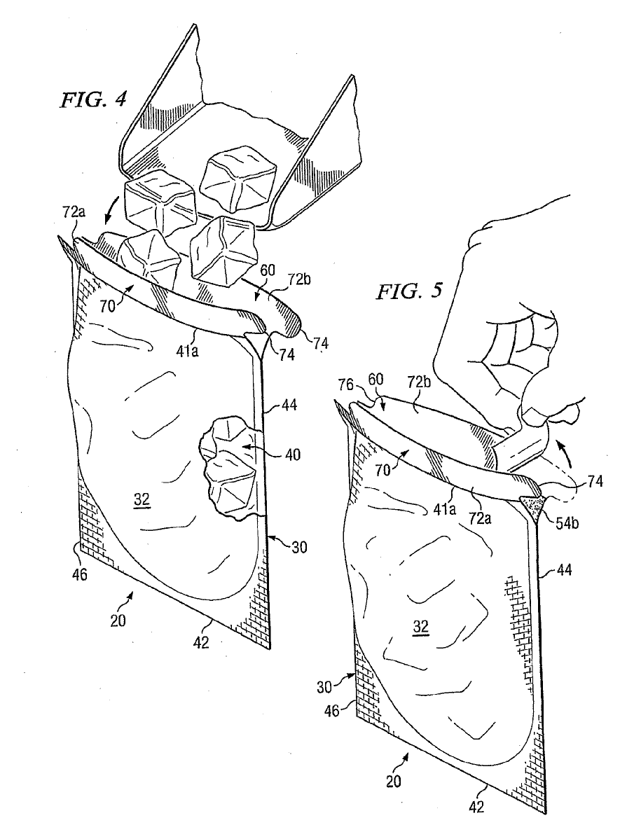

The release liners 72a and 72b can cooperate with each other to provide a disposable funnel-type device 70 to facilitate adding ice to the containment section 40. See FIG. 4. The release liners 72a and 72b may be formed from paper or other suitable plastic films. In addition to forming a disposable funnel-type device 70, the release liners 72a and 72b cooperate with each other to protect their associated adhesive layers 54a and 54b during fabrication of ice pack 20, storage prior to use, and while adding ice to containment section 40.

For some applications, a single adhesive layer 54 may be placed on an interior portion of either sidewall 34a or 34b adjacent to the opening 60. A single release liner 72 may be placed on the associated adhesive layer 54. However, using two adhesive layers 54a and 54b in combination with two release liners 72a and 72b will substantially improve the resulting fluid seal formed after ice has been placed within containment section 40. This seal is further enhanced by the two sealing surfaces created by making one adhesive layer of a lesser width than the other, as shown in FIGS. 2 and 3.

The bag 30 may be described as having a pair of “flaps” located adjacent to opening 60. The first flap includes the adhesive layer 54a, the edge 41a, the adjacent portions of associated layer 31 and the adjacent portions of associated layer 32. The second flap includes the adhesive layer 54b, the edge 41b, the adjacent portions of associated layer 31 and the adjacent portions of the associated layer 32. The first release liner 72a may be attached to the first flap. The second release liner 72b may be attached to the second flap.

For some applications portions of the fluid barrier 50 adjacent the respective edges 41a and 41b may have an increased width. For embodiments such as shown in FIG. 1, the increased width portions of the fluid barrier 50 have been designated as 52. The increased width portions 52 of the fluid barrier 50 cooperate with the adhesive layers 54a and 54b to form a fluid-tight seal in any gaps, that may be formed adjacent to the edges 41a, 41b and the other portions of the closure mechanism for the opening 60.

During fabrication of the ice pack 20, the release liner 72a and 72b may cause the formation of a gap between adjacent portions of sidewalls 34a and 34b proximate opening 60. The resulting gap may correspond approximately with the combined thickness of the release liner 72a and 72b. By using relatively viscous adhesive materials to form the adhesive layers 54a and 54b, any associated gaps will be generally filled when the adhesive layers 54a and 54b contact each other. Rubber-based pressure sensitive adhesive is one example of such viscous adhesive materials. As discussed later in more detail, combining the adhesive layers 54a and 54b with heat-sealing the periphery of the bag 30 substantially eliminates or reduces any gaps associated with closure of the bag 30 after it has been filled with ice.

Heat-sealing the lateral edges 44 and 46 with each other will form a fluid-tight barrier with adjacent portions of the first layer or inner layers 31. The fluid-tight barrier will terminate at the edge of the release liners 72a and 72b.

As a result, a small or slight gap may be formed equal to the combined thickness of the release liners 72a and 72b at the junction of the lateral edges 54 and 56 indicated as gap 56. See FIGS. 1 and 5.

For some applications, the release liners 72 may be formed or cut from a generally continuous strip of material (not expressly shown) with very little or no wasted material. For example, a rotary cutting machine may be used to form each release liner 72 having a generally elongated rectangular configuration and having a length corresponding approximately with the length of associated the adhesive layers 54. The width of each release liner 72 will often be larger than the width of associated adhesive layers 54a or 54b so as to form portions of disposable funnel type device 70. The width of the release ii liner 72 may correspond approximately with the width of associated adhesive layer 54.

For some applications, a tab 74 may be formed at one end of each release liner 72. A corresponding notch or recess 76 may be formed in the opposite end of each release liner 72. The dimensions and configuration of the tab 74 and the notch 76 may be selected to be compatible with each other. As a result, tab 74 of one ice pack 20 may be nested in respective notch 76 of an adjacent ice pack 20. Tabs 74 cooperate with each other to provide a convenient mechanism for removing associated release liners 72a and 72b after ice has been added to the containment section 40. Tabs 74 and notches 76 facilitate automated cutting of respective ice packs 20, with little or no material waste, from multiple layers of material disposed in an elongated strip (not expressly shown). The elongated strip may sometimes be referred to as a blank.

For some applications, multiple ice packs 20 may be fabricated in a substantially automated process using heat-sealing dies and rotary cutting elements (not expressly shown). For example, a first elongated sheet of laminated material compatible with forming sidewalls 34b may be placed on a relatively continuous fabrication line. Adhesive material satisfactory for forming adhesive layer 54b may be placed on one side of a first strip of material compatible with forming release liner 72b. Additional adhesive material satisfactory for forming adhesive layer 54a may be placed on one side of a second strip of material compatible with forming release liner 72a. The first strip of release liner and adhesive material may be placed on the first sheet of laminated material proximate the desired location for the edges 41b of resulting sidewalls 34b. The second strip of release liner and adhesive material may then be placed on the first strip of release liner with the associated adhesive material located opposite from the first strip of release liner (i.e., no adhesive material contact between the first strips and the second strip of release liners). A second elongated sheet of laminated material compatible with forming sidewalls 34a may then be placed on the second strip of release liner with the associated adhesive material displayed proximate the desired location for the edges 41a of resulting sidewalls 34a.

The resulting multiple layers of material form an elongated strip or blank. Rollers or other techniques may be used to move the blank through a heat-sealing die to form respective fluid barriers 50 in the blank corresponding with the periphery for each resulting bag 30. A rotary cutting element or other similar device may then be used to cut each ice pack 20 (bag 30 and release liners 72a and 72b) from the blank with little or no wasted material.

The formation of the bag 30 using heat-sealing techniques will preferably cause adhesive material used to form adhesive layers 54a and 54b to flow into adjacent flared portions 52 of fluid barrier 50 to enhance the sealing of any gaps disposed therein. See FIGS. 1 and 5. Placing the adhesive layers 54a and 54b adjacent to opening 60 prior to heat sealing bag 30 improves the resulting fluid tight barrier after the release liners 72a, 72b have been removed, and the adhesive layers 54a and 54b engaged with each other.

The adhesive layers 54a and 54b are generally disposed contiguous with and fill any gaps in adjacent portions of associated fluid barrier 50. For some applications, the adhesive layers 54a and 54b cooperate with each other to form a gasket type seal between adjacent portions of first layer 31 to form a permanent fluid tight barrier at opening 60.

Any variations in the configuration or dimensions of the release liners 72a and 72b, the adhesive layers 54a and 54b or adjacent portions of fluid barrier 50 will be compensated for by using heat-sealing techniques to form fluid barrier 50. The adhesive layers 54a and 54b are preferably formed from viscous material, which will flow into and fill any gaps. As a result, tolerance and dimensional requirements associated with forming opening 60 and the adhesive layers 54a and 54b may be reduced.

The design and configuration of the bag 30, fluid barrier 50 and release liners 72a and 72b cooperate with each other to allow the use of high-speed automated manufacturing techniques to produce the ice packs 20. The release liners 72a and 72b cooperate with each other to form a disposable funnel type device 70 for use in placing ice within the containment section 40. See FIG. 4.

For some applications, operating instructions or other information may be preprinted on the release liners 72a and 72b. The first release liner 72a and the second release liner 72b cooperate with each other to prevent ice or water contact with the adhesive layers 54a and 54b during filling of the containment section 40 with ice. After the containment section 40 has been filled with ice, release liners 72a and 72b may be removed from associated adhesive layers 54a and 54b. See FIG. 5.

The release liners 72a and 72b may be placed in a trash receptacle or other suitable disposal facility to further enhance sanitation and cleanliness associated with the use of the ice pack 20. The adhesive layers 54a and 54b may then be securely engaged with each other as well as with an interior portion of a sidewall 31b to form a fluid-tight barrier at the opening 60 to prevent water leaks from the containment section 40. Although the embodiments and their advantages have been described in detail, it should be understood that various changes, substitutions and alterations may be made without departing from the scope of the following claims.

I claim:

1. An ice pack comprising:
a first sidewall and a second sidewall;
the first sidewall and the second sidewall formed with at least one layer of waterproof material;
the first sidewall and the second sidewall bonded with each other to form a containment section;
an opening in communication with the containment section;
the opening defined in part by respective interior portions of the first sidewall and the second sidewall;
a first adhesive layer disposed adjacent to the opening and on an interior portion of the first sidewall;
a second adhesive layer disposed adjacent to the opening and on an interior portion of the second sidewall;
the second adhesive layer having a width less than that of the first adhesive layer, so that an exposed area of the interior of the second sidewall opposes the first adhesive layer;
the first adhesive layer and the second adhesive layer cooperating with each other to close the opening and form a fluid barrier in place of the opening when the first adhesive layer engages the second adhesive layer and the first adhesive layer further engages the exposed area of the interior of the second sidewall.

2. The ice pack of claim 1 further comprising each sidewall having a generally rectangular configuration.

3. The ice pack of claim 1 further comprising:
a first release liner disposed on the first layer of the adhesive material; and
a second release liner disposed on the second layer of adhesive material.

4. The ice pack of claim 3 further comprising the first release liner and the second release liner cooperating with each other to form a funnel type device.

5. The ice pack of claim 3 wherein each release liner further comprises:
a generally elongated rectangular configuration;
the generally elongated rectangular configuration defined in part by having a first end and a second end;
the first end of the release liner defined in part by a notch;
the second end of the release liner defined in part by an extended tab; and,
the configuration of the tab cooperating with the configuration of the notch whereby a series of release liners may be cut from a strip of material.

6. The ice pack of claim 1 wherein the layer of waterproof material is selected from the group consisting of polyethylene film, heat melt adhesive film, and waterproof nonwoven material.

7. The ice pack of claim 1 further comprising a second layer of material disposed on the layer of waterproof material.

8. The ice pack of claim 7, where the second layer of material is selected from the group consisting of hydroentangled fiber, hydroentangled wood pulp, absorbent polyester compounds, nonwoven fabric, and woven fabric.

9. The ice pack of claim 1 where the adhesive of the first adhesive layer and the adhesive of the second adhesive layer are selected from the group consisting of viscous adhesive material, acrylic based adhesives, rubber based adhesives and adhesive materials capable of filling any gap formed between the first sidewall and the second sidewall adjacent to the opening.

10. An ice pack comprising:
a first sidewall and a second sidewall;
the first sidewall and the second sidewall formed with at least a first layer of fluid impervious material and a second layer of soft, absorbent material;
the first sidewall and the second sidewall adhesively bonded with each other to form a containment section and further to form a continuous fluid barrier about a majority of a periphery of the containment section;
an opening defined in part by respective interior portions of the first sidewall and the second sidewall; the opening being in communication with the containment section;
a first adhesive layer disposed adjacent to the opening and on an interior portion of the first sidewall;
a second adhesive layer disposed adjacent to the opening and on an interior portion of the second sidewall;
the second adhesive layer having a width less than that of the first adhesive layer, so that an exposed area of the interior of the second sidewall opposes the first adhesive layer;
a first release liner disposed on the first layer of adhesive material;
a second release liner disposed on the second layer of adhesive material, so that the first release liner and the second release liner cooperate with each other to form a funnel at the opening; and,
the first adhesive layer and the second adhesive layer cooperating with each other when the first and second release liners are removed to close the opening and form a fluid barrier in place of the opening when the first adhesive layer engages the second adhesive layer and the first adhesive layer further engages the exposed area of the interior of the second sidewall.

11. The ice pack of claim 10 wherein the fluid barrier extends about the entire periphery of the containment section when the first adhesive layer is engaged with the second adhesive layer and the interior of the second sidewall.

12. The ice pack of claim 10 further comprising an enlarged portion of the fluid barrier disposed adjacent to the opening to accommodate any voids or gaps which may occur when the first adhesive layer engages the second adhesive layer to form a fluid barrier.
